SEQUENCE LISTING (41 sequences)

SEQ ID NO: 1 - DNA, 23 bp, Human Papillomavirus
  misc_feature (1)..(23): Molecule type synthetic DNA
aaacaatgacaacaactatgatg

SEQ ID NO: 2 - DNA, 25 bp, Human Papillomavirus
  misc_feature (1)..(25): Molecule type synthetic DNA
gaaaaataaactgtaaatcatattc

SEQ ID NO: 3 - DNA, 142 bp, Human Papillomavirus
  misc_feature (1)..(142): Molecule type genomic DNA (HPV type 16)
tttgttactgttgttgatactacacgcagtacaaatatgtcattatgtgctgccatatctacttcagaaactacatataaaaatactaactttaaggagtacctacgacatggggaggaatatgatttacagtttatttttc

SEQ ID NO: 4 - DNA, 18 bp, Human Papillomavirus
  misc_feature (1)..(18): Molecule type synthetic DNA
gctgccatatctacttca

SEQ ID NO: 5 - DNA, 18 bp, Human Papillomavirus
  misc_feature (1)..(18): Molecule type synthetic DNA
tgaagtagatatggcagc

SEQ ID NO: 6 - DNA, 145 bp, Human Papillomavirus
  misc_feature (1)..(145): Molecule type genomic DNA (HPV type 18)
tttgttactgtggtagataccactcccagtaccaatttaacaatatgtgcttctacacagtctcctgtacctgggcaatatgatgctaccaaatttaagcagtatagcagacatgttgaggaatatgatttgcagtttatttttc

SEQ ID NO: 7 - DNA, 16 bp, Human Papillomavirus
  misc_feature (1)..(16): Molecule type synthetic DNA
gtagcatcatattgcc

SEQ ID NO: 8 - DNA, 16 bp, Human Papillomavirus
  misc_feature (1)..(16): Molecule type synthetic DNA
ggcaatatgatgctac

SEQ ID NO: 9 - DNA, 142 bp, Human Papillomavirus
  misc_feature (1)..(142): Molecule type genomic DNA (HPV type 31)
tttgttactgtggtagataccacacgtagtaccaatatgtctgtttgtgctgcaattgcaaacagtgatactacatttaaaagtagtaattttaaagagtatttaagacatggtgaggaatttgatttacaatttatatttc

SEQ ID NO: 10 - DNA, 18 bp, Human Papillomavirus
  misc_feature (1)..(18): Molecule type synthetic DNA
gcaattgcaaacagtgat

SEQ ID NO: 11 - DNA, 18 bp, Human Papillomavirus
  misc_feature (1)..(18): Molecule type synthetic DNA
atcactgtttgcaattgc

SEQ ID NO: 12 - DNA, 139 bp, Human Papillomavirus
  misc_feature (1)..(139): Molecule type genomic DNA (HPV type 33)
tttgttactgtggtagataccactcgcagtactaatatgactttatgcacacaagtaactagtgacagtacatataaaaatgaaaattttaaagaatatataagacatgttgaagaatatgatctacagtttgtttttc

SEQ ID NO: 13 - DNA, 19 bp, Human Papillomavirus
  misc_feature (1)..(19): Molecule type synthetic DNA
atgcacacaagtaactagt

SEQ ID NO: 14 - DNA, 19 bp, Human Papillomavirus
  misc_feature (1)..(19): Molecule type synthetic DNA
actagttacttgtgtgcat

SEQ ID NO: 15 - DNA, 142 bp, Human Papillomavirus
  misc_feature (1)..(142): Molecule type genomic DNA (HPV type 35)
tttgttactgtagttgatacaacccgtagtacaaatatgtctgtgtgttctgctgtgtcttctagtgacagtacatataaaaatgacaattttaaggaatatttaaggcatggtgaagaatatgatttacagtttatttttc

SEQ ID NO: 16 - DNA, 18 bp, Human Papillomavirus
  misc_feature (1)..(18): Molecule type synthetic DNA
ctgctgtgtcttctagtg

SEQ ID NO: 17 - DNA, 18 bp, Human Papillomavirus
  misc_feature (1)..(18): Molecule type synthetic DNA
cactagaagacacagcag

SEQ ID NO: 18 - DNA, 145 bp, Human Papillomavirus
  misc_feature (1)..(145): Molecule type genomic DNA (HPV type 39)
tttcttactgttgtggacactacccgtagtaccaactttacattatctacctctatagagtcttccataccttctacatatgatccttctaagtttaaggaatataccaggcacgtggaggagtatgatttacaatttatatttc

SEQ ID NO: 19 - DNA, 21 bp, Human Papillomavirus
  misc_feature (1)..(21): Molecule type synthetic DNA
ctctatagagtcttccatacc

SEQ ID NO: 20 - DNA, 21 bp, Human Papillomavirus
  misc_feature (1)..(21): Molecule type synthetic DNA
ggtatggaagactctatagag

SEQ ID NO: 21 - DNA, 145 bp, Human Papillomavirus
  misc_feature (1)..(145): Molecule type genomic DNA (HPV type 45)
tttgttactgtagtggacactacccgcagtactaatttaacattatgtgcctctacacaaaatcctgtgccaagtacatatgaccctactaagtttaagcagtatagtagacatgtggaggaatatgatttacagtttatttttc

SEQ ID NO: 22 - DNA, 17 bp, Human Papillomavirus
  misc_feature (1)..(17): Molecule type synthetic DNA
ctacacaaaatcctgtg

SEQ ID NO: 23 - DNA, 17 bp, Human Papillomavirus
  misc_feature (1)..(17): Molecule type synthetic DNA
cacaggattttgtgtag

SEQ ID NO: 24 - DNA, 142 bp, Human Papillomavirus
  misc_feature (1)..(142): Molecule type genomic DNA (HPV type 51)
tttattacctgtgttgatactaccagaagtacaaatttaactattagcactgccactgctgcggtttccccaacatttactccaagtaactttaagcaatatattaggcatggggaagagtatgaattgcaatttatttttc

SEQ ID NO: 25 - DNA, 15 bp, Human Papillomavirus
  misc_feature (1)..(15): Molecule type synthetic DNA
cggtttccccaacat

SEQ ID NO: 26 - DNA, 15 bp, Human Papillomavirus
  misc_feature (1)..(15): Molecule type synthetic DNA
atgttggggaaaccg

SEQ ID NO: 27 - DNA, 139 bp, Human Papillomavirus
  misc_feature (1)..(139): Molecule type genomic DNA (HPV type 52)
tttgtcacagttgtggataccactcgtagcactaacatgactttatgtgctgaggttaaaaaggaaagcacatataaaaatgaaaattttaaggaataccttcgtcatggcgaggaatttgatttacaatttatttttc

SEQ ID NO: 28 - DNA, 17 bp, Human Papillomavirus
  misc_feature (1)..(17): Molecule type synthetic DNA
gtgctgaggttaaaaag

SEQ ID NO: 29 - DNA, 17 bp, Human Papillomavirus
  misc_feature (1)..(17): Molecule type synthetic DNA
ctttttaacctcagcac

SEQ ID NO: 30 - DNA, 139 bp, Human Papillomavirus
  misc_feature (1)..(139): Molecule type genomic DNA (HPV type 56)
tttgttactgtagtagatactactagaagtactaacatgactattagtactgctacagaacagttaagtaaatatgatgcacgaaaaattaatcagtaccttagacatgtggaggaatatgaattacaatttgtttttc

SEQ ID NO: 31 - DNA, 19 bp, Human Papillomavirus
  misc_feature (1)..(19): Molecule type synthetic DNA
ctacagaacagttaagtaa

SEQ ID NO: 32 - DNA, 19 bp, Human Papillomavirus
  misc_feature (1)..(19): Molecule type synthetic DNA
ttacttaactgttctgtag

SEQ ID NO: 33 - DNA, 139 bp, Human Papillomavirus
  misc_feature (1)..(139): Molecule type genomic DNA (HPV type 58)
tttgttaccgtggttgataccactcgtagcactaatatgacattatgcactgaagtaactaaggaaggtacatataaaaatgataattttaaggaatatgtacgtcatgttgaagaatatgacttacagtttgtttttc

SEQ ID NO: 34 - DNA, 17 bp, Human Papillomavirus
  misc_feature (1)..(17): Molecule type synthetic DNA
aactaaggaaggtacat

SEQ ID NO: 35 - DNA, 17 bp, Human Papillomavirus
  misc_feature (1)..(17): Molecule type synthetic DNA
atgtaccttccttagtt

SEQ ID NO: 36 - DNA, 145 bp, Human Papillomavirus
  misc_feature (1)..(145): Molecule type genomic DNA (HPV type 59)
tttttaacagttgtagatactactcgcagcaccaatctttctgtgtgtgcttctactacttcttctattcctaatgtatacacacctaccagttttaaagaatatgccagacatgtggaggaatttgatttgcagtttatatttc

SEQ ID NO: 37 - DNA, 22 bp, Human Papillomavirus
  misc_feature (1)..(22): Molecule type synthetic DNA
ctactactctctattcctaatg

SEQ ID NO: 38 - DNA, 22 bp, Human Papillomavirus
  misc_feature (1)..(22): Molecule type synthetic DNA
cattaggaatagagagtagtag

SEQ ID NO: 39 - DNA, 145 bp, Human Papillomavirus
  misc_feature (1)..(145): Molecule type genomic DNA (HPV type 68)
tttcttactgttgtggataccactcgcagtaccaattttactttgtctactactactgaatcagctgtaccaaatatttatgatcctaataaatttaaggaatatattaggcatgttgaggaatatgatttgcaatttatatttc

SEQ ID NO: 40 - DNA, 19 bp, Human Papillomavirus
  misc_feature (1)..(19): Molecule type synthetic DNA
ctttgtctactactactga

SEQ ID NO: 41 - DNA, 19 bp, Human Papillomavirus
  misc_feature (1)..(19): Molecule type synthetic DNA
tcagtagtagtagacaaag

(19) United States
(12) Patent Application Publication
(10) Pub. No.: US 2009/0170064 A1
(43) Pub. Date: Jul. 2, 2009

(54) ISOTHERMAL SCREENING OF HUMAN PAPILLOMAVIRUS RELATED NUCLEIC ACIDS

(76) Inventor: Frank G Salinas, Wheaton, IL (US)

Correspondence Address: MCANDREWS HELD & MALLOY, LTD, 500 WEST MADISON STREET, SUITE 3400, CHICAGO, IL, 60661, US

(21) Appl. No.: 11/679,688

(22) Filed: Feb. 27, 2007

Related U.S. Application Data
(60) Provisional application No. 60/777,168, filed on Feb. 27, 2006.

(51) Int. Cl. C12Q 1/70; C12N 9/10

(52) U.S. Cl. 435/5; 435/193

(57) ABSTRACT

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases.

### RELATED APPLICATIONS

The present application is related to and claims priority from U.S. Provisional Patent Application Ser. No. 60/777,168, filed Feb. 27, 2006, the contents of which are hereby incorporated herein by reference in their entirety. Additionally, all cited references in the present application are hereby incorporated by reference in their entirety.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The presently described technology relates generally to the art of molecular diagnostics and more particularly to point-of-care diagnostic methods and materials. The diagnostic methods and materials of the presently described technology are suitable for a variety of uses including but not limited to the bedside or field diagnosis of infectious or noninfectious diseases. The present invention relates to human papillomaviruses and, in particular, it relates to oligonucleotides and other methods and reagents for detecting human papillomaviruses in a test sample. In particular, the present invention relates to methods and materials for the isothermal detection of oncogenic HPV types 16, 18, 31, 33, 35, 39, 45, 51, 52, 56, 58, 59 and 68.

Approximately seventy different human papillomavirus (HPV) types have been discovered. HPV is interesting from a diagnostic standpoint because several of the presently known HPV types have been linked to the development of cervical cancer. As with any form of cancer, early detection is critical to successfully treating the disease. Because certain HPV strains are associated with the development of cervical cancer, detecting HPV in an appropriate sample may provide the best means for the early detection of cervical cancer.

One of the main challenge is to intensify prevention efforts and develop a field based point-of-care molecular diagnostic system for the amplification and detection of HPV.

### BRIEF SUMMARY OF THE INVENTION

One object of the present invention is to provide a molecular diagnostic system comprising methods and materials for the isothermal detection and screening of nucleic acids. Still another object of the present invention is to provide a molecular diagnostic system comprising methods and reagents for the isothermal detection and screening of nucleic acids associated with but not limited to disease, disease predisposition, disease causative agents, and any combination or derivative thereof. A further object of the present invention is to provide a molecular diagnostic system comprising methods and materials for the isothermal detection and screening of nucleic acids associated with human papillomavirus.

One or more of the preceding objects, or one or more other objects which will become plain upon consideration of the present specification, are satisfied by the invention described herein.

One aspect of the invention, which satisfies one or more of the above objects, is a test kit having reagents for the isothermal detection of nucleic acids associated with but not limited to disease, disease predisposition, disease causative agents, and any combination or derivative thereof. Another aspect of the invention is a test kit comprising: a strand transferase component; a polymerase component; and one or more primers and/or probes complementary to one or more nucleic acids associated with but not limited to disease, disease predisposition, disease causative agents, and any combination or derivative thereof. One preferred aspect of the present invention is a test kit comprising: a reverse transcriptase, a strand transferase component; a DNA dependent DNA polymerase component; and one or more primers and/or probes complementary to one or more nucleic acids associated with human papillomavirus.

### BRIEF DESCRIPTION OF SEVERAL VIEWS OF THE FIGURES

FIG. 1 is a schematic view of one aspect of the isothermal DNA amplification system of the present invention employing one primer complementary to a target nucleic acid, a strand transferase, and a polymerase.

FIG. 2 is a schematic view of another aspect of the isothermal DNA amplification system of the present invention employing two primers complementary to opposite strands and flanking a target nucleic acid, a strand transferase and a polymerase.

### DETAILED DESCRIPTION OF THE INVENTION

The present invention provides methods and materials for the isothermal screening and detection of nucleic acids associated with but not limited to disease, disease predisposition, disease causative agents, and any combination or derivative thereof. As used herein, and without limitation, nucleic acid generally includes any size DNA, RNA, DNA/RNA hybrid, or analog thereof. The nucleic acid can be single stranded, double stranded, or a combination of single and double stranded. As used herein, and without limitation, disease generally includes an impairment of the normal state of the living animal or plant body or one of its parts that interrupts or modifies the performance of the vital functions, is typically manifested by distinguishing signs and symptoms, and is a response to environmental factors (as malnutrition, industrial hazards, or climate), to specific infective agents (as parasites, bacteria, or viruses), to inherent defects of the organism (as genetic anomalies), or to combinations or derivatives of these factors.

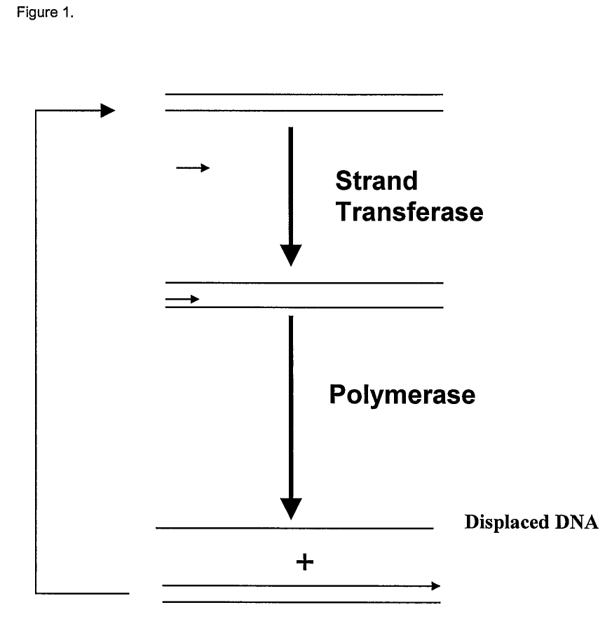

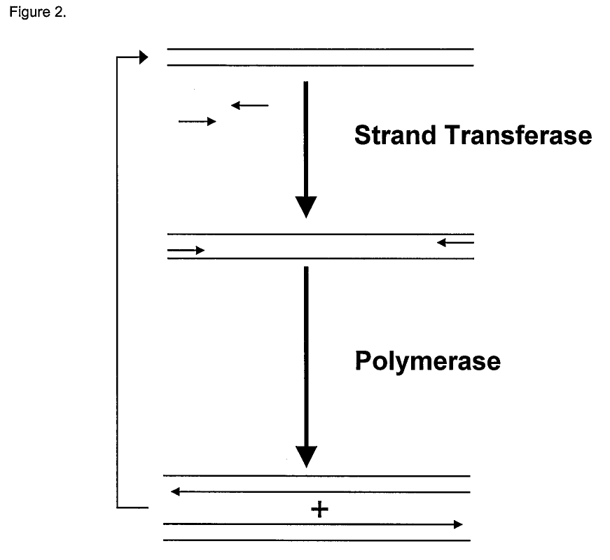

One aspect of the present invention includes methods and materials for the quantitative or qualitative isothermal screening and detection of one or more target nucleic acids of interest. This aspect of the present invention comprises contacting the target nucleic acid with at least one nucleic acid primer having complementarity to the target nucleic acid, a strand transferase, and a polymerase. The strand transferase catalyzes the homologous pairing of the at least one primer to a specific location on the target nucleic acid to form a primer-template junction that is acted upon by the polymerase to replicate and amplify the target nucleic acid (FIG. 1). In one preferred embodiment, the target nucleic acid is contacted with two primers complementary to opposite strands and flanking said target nucleic acid, in the presence of a strand transferase and a polymerase (FIG. 2). In certain aspects of the present invention, the isothermal amplification of the nucleic acid is performed as describe in U.S. Pat. No. 6,929,915, Methods for Nucleic Acid Manipulation. This reference is herein incorporated by reference.

As used herein without limitation, a strand transferase generally is a catalyst for the identification and base pairing of homologous sequences between nucleic acids, a process also known as homologous pairing or strand exchange. Bianco et al provides a general discussion of strand transferases in “DNA strand exchange proteins: a biochemical and physical comparison” at Front Biosci. 1998 Jun. 17; 3:D570-603. This reference is herein incorporated by reference. Strand transferases can be derived from either a prokaryotic system or an eukaryotic system, including but not limited to yeast, bacteria, and bacteriophages such as T4 and T7. For example West discusses eukaryotic strand transferases in Recombination genes and proteins” in Curr Opin Genet Dev. 1994 April; 4(2):221-8. This reference is herein incorporated by reference. Radding discussed the recA strand exchange protein in “Helical RecA nucleoprotein filaments mediate homologous pairing and strand exchange” at Biochim Biophys Acta. 1989 Jul. 7; 1008(2):131-45. This reference is herein incorporated by reference. Also, the UvsX strand transferase was described by Kodadek et al., The mechanism of homologous DNA strand exchange catalyzed by the bacteriophage T4 uvsX and gene 32 proteins” JBC 1988 Jul. 5; 263(19):9427-36. This reference is herein incorporated by reference. Yonesaki discusses T4 homologous recombination in “Recombination apparatus of T4 phage” at Adv Biophys. 1995; 31:3-22. This reference is herein incorporated by reference. Also, Salinas et. al have discussed the homology dependence of UvsX catalyzed strand exchange in “Homology dependence of UvsX protein-catalyzed joint molecule formation” at J Biol. Chem. 1995 Mar. 10; 270(10):5181-6. This reference is herein incorporated by reference. Exemplar strand transferase proteins include but are not limited to the eukaryotic Rad51 protein, the bacterial recA protein, the bacterial phage T4 UvsX protein, the bacteriophage T7 gene 2.5 or any protein fragment, derivative, or homolog thereof, including proteins found in nature and those engineered or modified using recombinant DNA technology. Kong et. al has discussed T7 strand exchange in “Role of the bacteriophage T7 and T4 single-stranded DNA-binding proteins in the formation of joint molecules and DNA helicase-catalyzed polar branch migration.” J Biol. Chem. 1997 Mar. 28; 272(13):8380-7. This reference is herein incorporated by reference.

Strand transferases generally operate by first binding single stranded regions of DNA to form a nucleoprotein filament generally referred to as the presynaptic filament. The presynaptic filament then binds a target nucleic acid and performs a search for homology that once complete results in the formation of a joint molecule or D-loop. Strand transferases generally have accessory protein factors that augment or modify their activity. For example, strand transferases generally have accessory protein factors that effect the formation and/or stability of the presynaptic filament under varying conditions, including for example buffer conditions and/or the presence of other proteins competing to bind regions of single-stranded nucleic acid. Exemplar strand transferase accessory proteins include but are not limited to the bacteriophage T4 UvsX accessory protein UvsY, the E. coli RecA accessory proteins RecFOR, the yeast and human Rad51 accessory protein Rad52, and any protein fragment, derivative, or homolog thereof, including proteins found in nature and those engineered or modified using recombinant DNA technology.

As used herein without limitation, a polymerase generally is any of several enzymes, such as DNA polymerase, RNA polymerase, or reverse transcriptase, that catalyze the formation of nucleic acid from precursor substances in the presence of preexisting nucleic acid acting as a template. The polymerase of the present invention can be derived from a eukaryotic or a prokaryotic system. For example the polymerase can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar polymerases include but are not limited to the bacteriophage T4 gene product 43 protein, and any mutants or derivatives of the gene 43 protein including but not limited to the exonuclease deficient 43 exo⁻ polymerase. Benkovic et. al discusses replisome mediated DNA replication in “Replisome Mediated DNA Replication” at Annu Rev Biochem. 2001; 70:181-208. This reference is herein incorporated by reference.

Polymerases generally have accessory protein factors that augment or modify their activity. Exemplar polymerase accessory factors include but are not limited to clamp proteins and clamp loader proteins. Clamp proteins generally have affinity and/or a topological link to both the polymerase and the nucleic acid being acted upon by said polymerase, thereby forming a stable link between polymerase and nucleic acid, the result of which is the formation of a stable polymerase nucleic acid complex having high processivity Clamp loader proteins facilitate the assembly of a clamp protein onto a nucleic acid and can also facilitate and mediate a concomitant or subsequent interaction with the polymerase. As used herein in connection with certain aspects and embodiments of the invention, the term holoenzyme generally regards a polymerase-clamp complex.

Polymerase accessory factors can be derived from a bacterium such as E. coli, a bacteriophage such as bacteriophage T4 or bacteriophage T7, a eukaryotic organism such as yeast or human, a virus, or any protein fragment, derivative, or homolog thereof, including proteins found in nature and those engineered or modified using recombinant DNA technology. Exemplar clamp proteins include but are not limited to the bacteriophage T4 gene product 45 protein, and any mutants or derivatives of the T4 gene product 45 protein. Trakselis et discuss the T4 polymerase holoenzyme in Creating a dynamic picture of the sliding clamp during T4 DNA polymerase holoenzyme assembly by using fluorescence resonance energy transfer” at Proc Natl Acad Sci USA. 2001 Jul. 17; 98(15):8368-75. This reference is herein incorporated by reference.

In certain embodiments of the present invention, the quantitative or qualitative isothermal screening and detection of one or more target nucleic acids of interest is performed in the presence of a single stranded nucleic acid binding protein (SSB). SSB's used pursuant to the present invention can be derived from a bacterium such as E. coli, a bacteriophage such as bacteriophage T4 or bacteriophage T7, a eukaryotic organism such as yeast or human, or any protein fragment, derivative, or homolog thereof, including proteins found in nature and those engineered or modified using recombinant DNA technology. Exemplar SSB's include but are not limited to the E. coli SSB protein, the bacteriophage T4 gene product 32 protein, the bacteriophage T7 gene product 2.5 protein, and the yeast or human RPA protein, or any mutants or derivatives thereof.

In certain embodiments of the present invention, the quantitative or qualitative isothermal screening and detection of one or more target nucleic acids of interest is performed in the presence of a helicase, preferably a DNA helicase. The helicase can be derived from a prokaryote or a eukaryote. For example, the DNA helicase can be from a bacterium such as E. coli., a bacteriophage such as bacteriophage T4 or bacteriophage T7, a yeast, or human. Exemplar helicases include but are not limited to the bacteriophage T4 gene product 41, the bacteriophage T4 dda protein, the bacteriophage T7 gene 4 protein, the E. coli UvrD protein, and any mutants or derivatives thereof. For example, Salinas and Kodadek have discussed the role of DNA helicases during strand homologous recombination in “Phage T4 homologous strand exchange: a DNA helicase, not the strand transferase, drives polar branch migration.” Cell 1995 Jul. 14; 82(1):111-9. This reference is herein incorporated by reference. Also, Salinas and Benkovic have discussed the role of DNA helicases in bacteriophage T4 replication in “Characterization of bacteriophage T4-coordinated leading- and lagging-strand synthesis on a minicircle substrate.” Proc Natl Acad Sci USA. 2000 Jun. 20; 97(13):7196-201. This reference is herein incorporated by reference. Also, Alberts et al discusses the general nature of replication in bacteriophage T4 in “Studies on DNA replication in the bacteriophage T4 in vitro system” at Cold Spring Harb Symp Quant Biol. 1983; 47 Pt 2:655-68. This reference is herein incorporated by reference.

In certain other embodiments of the present invention, the quantitative or qualitative isothermal screening and detection of one or more target nucleic acids of interest is performed in the presence of a helicase and a helicase accessory factor. The DNA helicase and the DNA helicase accessory factor can be derived from a eukaryotic or prokaryotic system. For example, the DNA helicase and the DNA helicase accessory factor can be from a bacterial system such as E. coli. or a bacteriophage system such as bacteriophage T4. For example, one DNA helicase/accessory factor pair is the bacteriophage T4 gene product 41 protein and its accessory factor gene product 59 protein. Jones et al discusses the gene product 59 protein in “Bacteriophage T4 gene 41 helicase and gene 59 helicase-loading protein: a versatile couple with roles in replication and recombination” at Proc Natl Acad Sci USA. 2001 Jul. 17; 98(15):8312-8. This reference is herein incorporated by reference.

In still other embodiments of the present invention, the quantitative or qualitative isothermal screening and detection of one or more target nucleic acids of interest is performed in the presence of a primosome. As used herein a primosome is a term that generally characterizes a complex comprising a DNA helicase and an RNA polymerase usually referred to as a primase. The primosome is active in synthesizing RNA primers on the lagging strand of a replication fork for the initiation of Okazaki fragment synthesis during coordinated leading- and lagging strand synthesis. Primases can be derived from a prokaryote or a eukaryote. For example, the primase can be from a bacterium such as E. coli., a bacteriophage such as bacteriophage T4 or bacteriophage T7, a yeast, or a human. One exemplar primase is the bacteriophage T4 gene product 61 protein, and derivatives or mutants thereof.

The phrase “amplification reaction reagents” as used herein includes but is not limited to reagents which are well known for their use in nucleic acid amplification reactions and may include but are not limited to: a single or multiple reagent, reagents, enzyme or enzymes separately or individually having reverse transcriptase and/or polymerase activity, strand transferase activity, or exonuclease activity; enzyme cofactors such as magnesium or manganese; salts; nicotinamide adenine dinucleotide (NAD); and deoxynucleoside triphosphates (dNTPs) such as, for example, deoxyadenosine triphosphate, deoxyguanosine triphosphate, deoxycytodine triphosphate and thymidine triphosphate. Other reagents include molecular crowding agents, including but not limited to polyethylene glycol PEG 8000. The exact amplification reagents employed are largely a matter of choice for one skilled in the art based upon the particular amplification reaction employed. For example, it is known in the art that volume occuping agents, or molecular crowding agents, inhance the activity or function of strand transferases, polymerases, and their accessory factors. The following references are herein incorporated by reference: (1) “Enhancement of recA Protein-promoted DNA Strand Exchange Activity by Volume occupying agents” at J Biol. Chem. 1992 May 5; 267(13):9307-14; (2) “Stimulation of the processivity of the DNA polymerase of bacteriophage T4 by the polymerase accessory proteins” at J Biol. Chem. 1991 Jan. 25; 266(3):1830-40; (3) “Macromolecular crowding”: thermodynamic consequences for protein-protein interactions within the T4 DNA replication complex: The role of ATP hydrolysis”; (4) “Macromolecular crowding”: thermodynamic consequences for protein-protein interactions within the T4 DNA replication complex” at J Biol. Chem. 1990 Sep. 5; 265(25):15160-7; (5) “Assembly of a functional replication complex without ATP hydrolysis: a direct interaction of bacteriophage T4 gp45 with T4 DNA polymerase” at Proc Natl Acad Sci USA. 1993 Apr. 15; 90(8):3211-5; and (6) “A coupled complex of T4 DNA replication helicase (gp41) and polymerase (gp43) can perform rapid and processive DNA strand-displacement synthesis” at Proc Natl Acad Sci USA. 1996 Dec. 10; 93(25):14456-61.

### Target Nucleic Acids

Target nucleic acids of the present invention include but are not limited to those nucleic acids associated with the development or onset of a disease state, including for example those nucleic acids that show the presence of specific infective agents or inherent defects of in an organism's genome. Target nucleic acids include but are not limited to nucleic acids that are exogenous and/or endogenous to the organism being screened. Exemplar target nucleic acids belonging to specific infective agents of interest include but are not limited to those nucleic acids derived from protozoa, parasites, fungi, bacteria, viruses, and combinations or derivatives thereof.

An object of the present invention is to provide methods and materials for the isothermal detection and screening of nucleic acids associated with human papillomavirus. Primerand probes for the amplification and detection of nucleic acids associated with the human papillomavirus (HPV) have been described in U.S. Pat. No. 6,265,154, Nucleic acid primers and probes for detecting oncogenic human papillomaviruses. This reference is herein incorporated by reference.

The present invention provides oligonucleotides that can be used in accordance with the isothermal DNA amplification technology described herein to specifically detect oncogenic HPV types 16, 18, 31, 33, 35, 39, 45, 51, 52, 56, 58, 59 and 68 (hereinafter “oncogenic HPV types”). These oligonucleotides are designated SEQ ID NO 4 and its complement SEQ ID NO 5; SEQ ID NO 7 and its complement SEQ ID NO 8; SEQ ID NO 10 and its complement SEQ ID NO 11; SEQ ID NO 13 and its complement SEQ ID NO 14; SEQ ID NO 16 and its complement SEQ ID NO 17; SEQ ID NO 19 and its complement SEQ ID NO 20; SEQ ID NO 22 and its complement SEQ ID NO 23; SEQ ID NO 25 and its complement SEQ ID NO 26; SEQ ID NO 28 and its complement SEQ ID NO 29; SEQ ID NO 31 and its complement SEQ ID NO 32; SEQ ID NO 34 and its complement SEQ ID NO 35; SEQ ID NO 37 and its complement SEQ ID NO 38; as well as SEQ ID NO 40 and its complement SEQ ID NO 41. Preferred are cocktails of these probes comprising two or more of the above oligonucleotides.

Preferably, the oligonucleotides are employed as hybridization probes to hybridize with and detect target sequences for which they are specific. Thus, methods provided by the present invention include hybridization assays as well as amplification based assays. According to one method, a method of detecting the presence of at least one oncogenic HPV type in a test sample comprises the steps of (a) contacting the test sample with one or more of the sequences listed above; and (b) detecting hybridization between at least one of the above sequences and an oncogenic HPV target sequence as an indication of the presence of at least one oncogenic HPV type in the test sample.

According to another embodiment, a method for detecting the presence of at least one oncogenic HPV type in a test sample comprises the steps of (a) forming a reaction mixture comprising a strand transferase, a polymerase, optionally a reverse transcriptase, a test sample containing an oncogenic HPV target sequence, at least one (and preferably two) primer(s) capable of amplifying an HPV target sequence designated herein as SEQ ID NO.3, SEQ ID NO. 6, SEQ ID NO. 9, SEQ ID NO. 12, SEQ ID NO. 15, SEQ ID NO. 18, SEQ ID NO. 21, SEQ ID NO. 24, SEQ ID NO. 27, SEQ ID NO. 30, SEQ ID 35 NO. 33, SEQ ID NO. 36, and SEQ ID NO. 39 and one or more oligonucleotides selected from the group consisting of SEQ ID NO 4, SEQ ID NO 7, SEQ ID NO. 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34, SEQ ID NO 37, SEQ ID NO 40, and their respective complements; (b) subjecting the mixture to hybridization conditions to produce at least one nucleic acid sequence complementary to the target sequence; (c) hybridizing one or more oligonucleotides to the nucleic acid sequence complementary to the target sequence, so as to form at least one complex comprising the oligonucleotide and the complementary nucleic acid sequence; and (d) detecting the so-formed complex as an indication of the presence of at least one oncogenic HPV type in the sample.

According to another embodiment, the invention provides kits which comprise a set of oligonucleotide primers, a strand transferase, at least one DNA dependent DNA polymerase, optionally at least one reverse transcriptases, and at least one, and preferably at least two, of the oligonucleotides designated as SEQ ID NO. 4, SEQ ID NO. 5, SEQ ID NO. 7, SEQ ID NO. 8, SEQ ID NO. 10, SEQ ID NO. 11, SEQ ID NO. 13, SEQ ID NO. 14, SEQ ID NO. 16, SEQ ID NO. 17, SEQ ID NO. 19, SEQ ID NO. 20, SEQ ID NO. 22, SEQ ID NO. 23, SEQ ID NO. 25, SEQ ID NO. 26, SEQ ID NO. 28, SEQ ID NO. 29, SEQ ID NO. 31, SEQ ID NO. 32, SEQ ID NO. 34, SEQ ID NO. 35, SEQ ID NO. 37, SEQ ID NO. 38, SEQ ID NO. 40 and SEQ ID NO. 41.

What is claimed is:

1. A method for detecting the presence of human papillomavirus nucleic acid in a test sample comprising contacting a test sample with a strand transferase, a polymerase, and at least one primer having complementarity to said human papillomavirus nucleic acid.

2. The method of claim 1 wherein said strand transferase is derived from a prokaryotic.

3. The method of claim 1 wherein said strand transferase is the uvsX strand transferase derived from the bacteriophage T4.

4. The polymerase of claim 1 wherein said polymerase is derived from a prokaryotic.

5. The polymerase of claim 1 wherein said polymerase is the gp43 polymerase derived from the bacteriophage T4.
